(19) United States
(12) Patent Application Publication
(10) Pub. No.: US 2011/0201383 A1
(43) Pub. Date: Aug. 18, 2011

(54) SENSOR APPARATUS AND SENSOR SYSTEM USING THE SAME

(75) Inventor: Hsiung HSIAO, HsinChu City (TW)

(73) Assignee: Middleland Sensing Technology Inc., HsinChu City (TW)

(21) Appl. No.: 13/005,191

(22) Filed: Jan. 12, 2011

Related U.S. Application Data
(63) Continuation-in-part of application No. 12/775,518, filed on May 7, 2010.

(30) Foreign Application Priority Data
Feb. 12, 2010 (TW) 099104639

(51) Int. Cl. H04M 1/00; G08C 19/16; A61B 5/00

(52) U.S. Cl. 455/556.1; 340/870.01; 600/309; 600/365; 600/364

(57) ABSTRACT

A sensor apparatus includes a portable electronic device and a sensor unit. The portable electronic device includes a signal processing unit, a transmitting unit, a receiving unit and an outputting unit. The sensor unit is adapted to electrically connecting with the portable electronic device by a USB interface. The sensor unit is adapted for sensing a physical and/or chemical parameter of a target object and generating a digital signal. The signal processing unit is capable of receiving the digital signal via the USB interface and then transmitting the digital signal to the transmitting unit. The transmitting unit transmits the digital signal to a server. The server processes the digital signal for obtaining a result. The receiving unit is capable of receiving the result from the server. The outputting unit is electrically connected with the receiving unit and adapted to output the result.

### CROSS-REFERENCE TO RELATED APPLICATIONS

This application is a continuation in part application of U.S. patent application Ser. No. 12/775,518 filed on May 7, 2010, which claims the foreign priority of Taiwanese patent application 099104639 filed on Feb. 12, 2010.

### FIELD OF THE INVENTION

The present invention relates to a sensor apparatus, and more particularly to a sensor apparatus and a sensor system using the sensor apparatus with a detachable and interchangeable sensor unit.

### BACKGROUND OF THE INVENTION

A conventional portable sensor apparatus generally includes a sensor unit, a display unit and a signal processing unit. The sensor unit has a sensor probe and is for measuring or monitoring physical and chemical parameters of a target object. The signal processing unit is configured to process signals obtained from the sensor unit and sometimes, to analyze and to store the resulted data. The display unit has a display screen and is for displaying results of the sensing process. During the measuring process, the user can visualize acquired data displayed on screen.

However, a portable sensor apparatus usually has the sensor unit, the processing unit, and the display unit integrally built, i.e., different types of the sensor units are coupled with different types of processing units and display units respectively, which are not interchangeable. The function of a portable sensor apparatus is hence limited by the type of sensor probes attached, which results in limited applications of a single sensor apparatus. When multiple analyses are involved, such as chemical compositions of both liquid samples and gas samples, different portable sensor apparatuses are usually required, which inevitably increases cost and reduces mobility, and hence results in inconvenience for users.

### SUMMARY OF THE INVENTION

Accordingly, the present invention is directed to a sensor apparatus with a detachable and interchangeable sensor unit that can be detached from and installed onto a portable electronic device, which can be used to measure or detect different physical and/or chemical parameters conveniently.

The present invention provides a sensor apparatus, which includes a portable electronic device and a sensor unit. The portable electronic device includes a processing unit, a transmitting unit, a receiving unit and an outputting unit. The sensor unit is adapted to electrically connecting with the portable electronic device by a USB interface. The sensor unit is adapted for measuring and monitoring a physical and/or chemical parameter of a target object and generating a digital signal. The signal processing unit is capable of receiving the digital signal via the USB interface and then transmitting the digital signal to the transmitting unit. The transmitting unit transmits the digital signal to a server. The receiving unit is capable of receiving a result obtained by the server which is for processing the digital signal. The outputting unit is electrically connected with the receiving unit and adapted to output the result.

The present invention also provides a sensor system, which includes a sensor unit, a server and a portable electronic device. The sensor unit is adapted for measuring and monitoring a physical and/or chemical parameters of a target object and generating a digital signal. The portable electronic device is adapted to electrically connecting with sensing unit by a USB interface. The portable electronic device includes a signal processing unit, a transmitting unit, a receiving unit and an outputting unit. The signal processing unit is capable of receiving the digital signal via the USB interface and then transmitting the digital signal to the transmitting unit. The transmitting unit delivers the digital signal to the server. The server processes the digital signal to obtain a result and transmits the result to the receiving unit. The outputting unit is electrically connected with the receiving unit and adapted to output the result.

In above sensor apparatus or sensor system, the sensor unit is detachably assembled with the portable electronic device. Therefore, if the some different physical and/or chemical datum need be measured, only one portable electronic device and some different sensing units should be carried. As such, volume, weight and cost of the various components that should be carried can be reduced, and thus the sensor system can be used conveniently.

### BRIEF DESCRIPTION OF THE DRAWINGS

The above objects and advantages of the present invention will become more readily apparent to those ordinarily skilled in the art after reviewing the following detailed description and accompanying drawings, in which:

FIG. 1 is a schematic diagram of a sensor system in accordance with a first embodiment of the present invention.

FIG. 2 is a schematic diagram of a sensor apparatus consisting of a portable electronic device and a sensor unit in the sensor system of FIG. 1.

FIG. 3 is a schematic diagram of a sensor system in accordance with a second embodiment of the present invention.

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

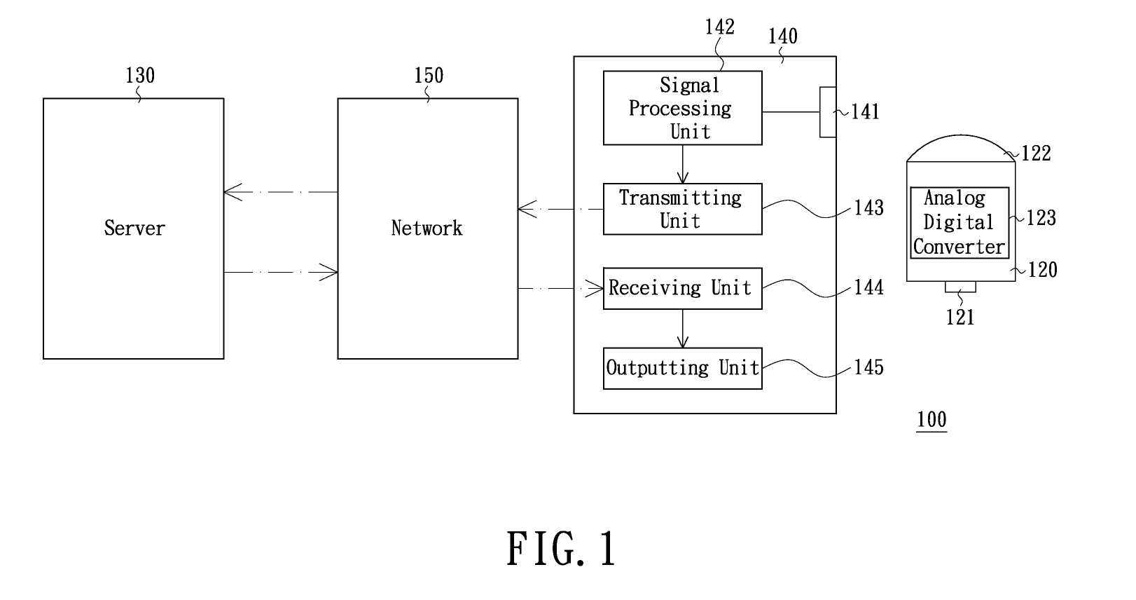

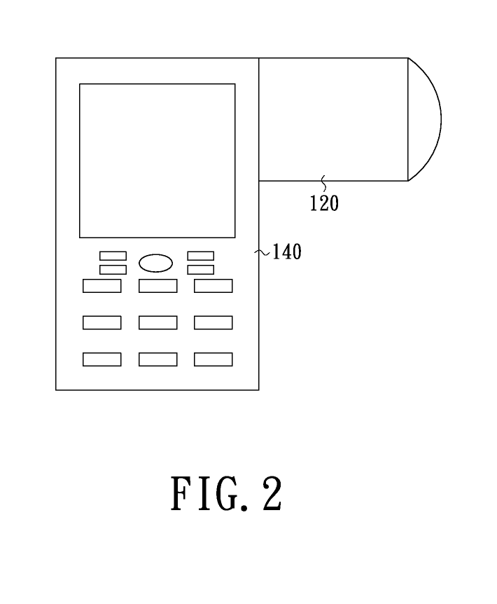

FIG. 1 is a schematic diagram of a sensor system in accordance with a first embodiment of the present invention. FIG. 2 is a schematic diagram of a sensor apparatus consisting of a portable electronic device and a sensor unit in the sensor system of FIG. 1. Referring to FIG. 1, the sensor system 100 includes a sensor unit 120, a server 130 and a portable electronic device 140. The sensor unit 120 is used for measuring and monitoring physical and/or chemical parameters of a target object (not shown) and generating a digital signal. The portable electronic device 140 can be coupled with the different sensor units 120 and transmit the digital signal to the server 130. The server 130 can process the digital signal to obtain a result and transmit the result back to the portable electronic device 140. Finally, the portable electronic device 140 can output the result.

Referring to FIG. 2, the sensor unit 120 and the portable electronic device 140 can electrically connect with each other to form a sensor apparatus by a universal series bus (USB) interface. The USB interface includes a USB plug 121 and a USB port 141. In the embodiment, the USB plug 121 is disposed on the sensor unit 120, and the USB port 141 is disposed on the portable electronic device 140.

The USB plug 121 on the sensor unit 120 can be plugged in the USB port 141 on the portable electronic device 140, so that the sensor unit 120 can electrically connect to the portable electronic device 140, and the digital data can be transmitted between the sensor unit 120 and the portable electronic device 140.

The sensor unit 120 further includes a sensor probe 122 and an analog digital converter 123. The sensor probe 122 is used for measuring a physical and/or chemical parameter of a target object to generate an analog signal. The analog signal can be a micro-voltage signal or a micro-current signal. The micro-voltage signal or the micro-current signal can be converted into the digital signal by the analog digital converter 123.

In details, the target object can be liquid, gas or solid, such as human body fluid. Types of the sensor unit 120 and the physical and/or chemical parameters measured by the sensor unit 120 would be various according to the different sensor probes 122. In the embodiment, the sensor unit 120 can be a blood glucose sensor, an ion sensor, a lipid sensor, a urea nitrogen sensor or other biological sensors for the body fluid. The sensor unit 120 can also be an optical sensor, such as a refractometer or a blood oxygen sensor. However, the present invention is not limited herein. Corresponding to the type of the sensor unit 120, the physical and/or chemical data can relate to blood glucose concentration, blood glucose level, lipid content, ion content in body fluid, blood oxygen content or urea nitrogen content or other biological parameters of the body fluid.

In the embodiment, the portable electronic device 140 can be a mobile phone, which includes a signal processing unit 142, a transmitting unit 143, a receiving unit 144 and an outputting unit 145. The processing unit 142 is electrically connected with the sensing unit 120 to receive the digital signal via the USB port 141 and the USB plug 121 and then transmit the digital signal to the transmitting unit 143. The transmitting unit 143 transmits the digital signal to the server 130. The server 130 processes the digital signal to obtain a result and then transmits the result back to the receiving unit 144. The result can include a physical and/or chemical parameter of the target object. The outputting unit 145 is electrically connected with the receiving unit 144 to output the result.

In details, the transmitting unit 143 transmits the digital signal to the server 130 by a network 150. The network 150 can be internet or intranet. Transmission mode of the network 150 can be wireless transmission or cable transmission. The wireless transmission can include GPRS, WIFI, 3G, WiMAX or LTE. The cable transmission can be achieved by using RJ45 cables or telephone lines. In the embodiment, the server 130 can also transmit the result back to the receiving unit 144 by the network 150. The outputting unit 145 can be a display device and the result is displayed by the outputting device 145 accordingly. Particularly, the result can further include a result response message, an advertising message or medical resource information except for the physical and/or chemical parameter of the target object. The result response message can be some advices corresponding to the physical and/or chemical parameter. For example, if the target object is blood glucose and the blood glucose level is high, the result response message can include description of the high blood glucose, a list of drugs or health products for regulating blood glucose, and the present invention is not limited herein. The advertising message can be some information related to the physical and/or chemical parameter that is provided for reference, and the present invention is not limited herein.

In the embodiment, the sensor unit 120 can be assembled with the portable electronic device 140 detachably and interchangeably. Therefore, in the sensor system 100, if the some different physical and/or chemical datum need be measured, only one portable electronic device 140 and some different sensor units 120 should be carried. As such, volume and weight of the various components that should be carried can be reduced, and thus the sensing system 100 can be used conveniently.

Furthermore, only one portable electronic device 140 and some different sensor units 120 should be carried while some different physical and/or chemical datum are measured by the sensing system 100, therefore cost for buying different electronic devices can be reduced, and thus the cost of sensing work can be reduced.

In addition, because the all digital signals can be transmitted to the server 130 to process, the structures of the sensor apparatus including the sensor unit 120 and the portable electronic device 140 can be simplified. Therefore, the cost of the sensor apparatus can be reduced. The all digital signals are processed by the server 130, therefore processing programs or other application programs can be easily updated in the server 130, and thus the application range of the sensor apparatus can be further expanded.

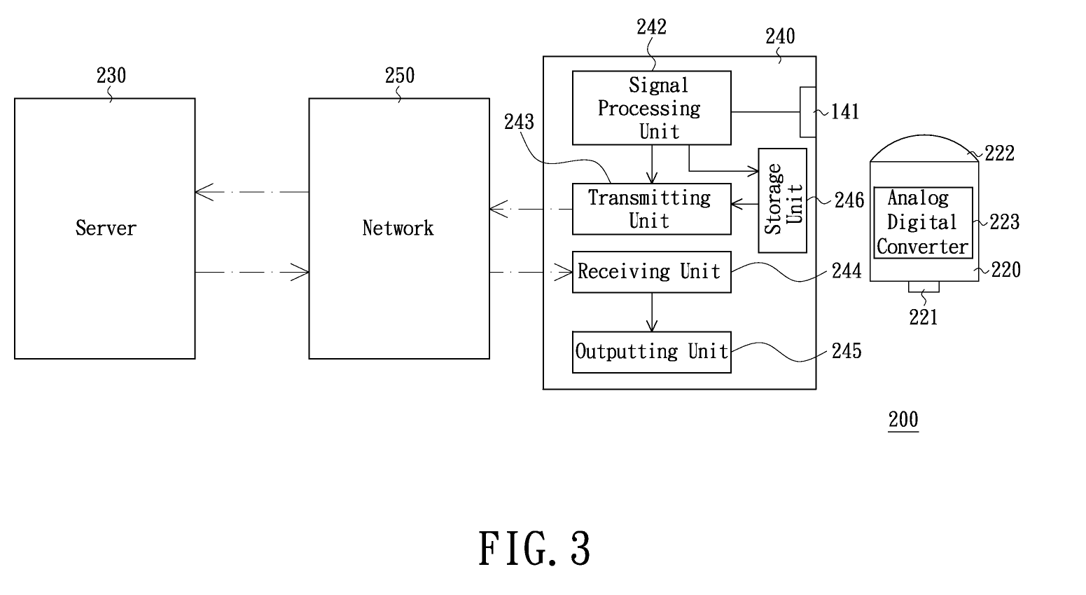

FIG. 3 is a schematic diagram of a sensor system in accordance with a second embodiment of the present invention. Referring to FIG. 3, the difference between the sensor system 200 of the second embodiment and the sensor system 100 of the first embodiment lies in a portable electronic device 240 with a storage unit 246. In the embodiment, a sensor unit 220 includes a sensor probe 222 and an analog digital converter 223. The sensor probe 222 is used for measuring a physical and/or chemical data of a target object to generate an analog signal. The analog signal can be a micro-voltage signal or a micro-current signal. The micro-voltage signal or the micro-current signal can be converted into a digital signal by the analog digital converter 223. The portable electronic device 240 further includes the storage unit 246 electrically connected with a signal processing unit 242 and used to store the digital signal. The storage unit 246 can be electrically connected with a transmitting unit 243, so that the stored digital signal can be transmitted to a server 230 to process by the transmitting unit 243 while needed.

As mentioned above, each of the embodiments of the present invention at least has one of the following advantages or other advantages.

First, the sensor unit is detachably and interchangeably assembled with the portable electronic device. Therefore, if the some different physical and/or chemical datum need be measured, only one portable electronic device and some different sensor units should be carried. As such, volume and weight of the various components that should be carried can be reduced, and thus the sensing system can be used conveniently.

Second, only one portable electronic device and some different sensor units should be carried while some different physical and/or chemical datum are measured by the sensor system, therefore cost for buying different electronic devices can be reduced, and thus the cost of sensing work can be reduced.

Third, because the all digital signal can be transmitted to the server to process, the structures of the sensor apparatus consisting of the sensing unit and the portable electronic device can be simplified. Therefore, the cost of the sensor apparatus can be reduced. The all digital signals are processed by the server, therefore processing programs or other application programs can be easily updated in the server, and thus the application range of the sensor apparatus can be further expanded.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A sensor apparatus, comprising:
a portable electronic device including a signal processing unit, a transmitting unit, a receiving unit and an outputting unit; and
a sensor unit adapted to electrically connecting with the portable electronic device by a USB interface, the sensing unit adapted for measuring a physical and/or chemical parameter of a target object and generating a digital signal, wherein the signal processing unit being capable of receiving the digital signal via the USB interface and then transmitting the digital signal to the transmitting unit, the transmitting unit transmitting the digital signal to a server, the server processing the digital signal for obtaining a result, the receiving unit be capable of receiving the result obtained from the server, and the outputting unit being electrically connected with the receiving unit and adapted to output the result.

2. The sensor apparatus according to claim 1, wherein the transmitting unit transmits the digital signal to the server by a network.

3. The sensor apparatus according to claim 1, wherein the result comprises a result response message or an advertising message.

4. The sensor apparatus according to claim 1, wherein the portable electronic device is a mobile phone.

5. The sensor apparatus according to claim 1, wherein the portable electronic device further comprises a storage unit electrically connected with the processing unit and adapted to store the digital signal.

6. A sensor system, comprising:
a sensor unit adapted for sensing a physical and/or chemical data of a target object and generating a digital signal;
a server; and
a portable electronic device adapted to electrically connecting with sensor unit by a USB interface, the portable electronic device including a processing unit, a transmitting unit, a receiving unit and an outputting unit, the processing unit being capable of receiving the digital signal via the USB interface and then transmitting the digital signal to the transmitting unit, the transmitting unit transmitting the digital signal to the server, the server processing the digital signal to obtain a result and transmitting the result to the receiving unit, and the outputting unit being electrically connected with the receiving unit and adapted to output the result.

7. The sensor system according to claim 6, wherein the transmitting unit transmits the digital signal to the server by a network.

8. The sensor system according to claim 6, wherein the result comprises a result response message or an advertising message.

9. The sensor system according to claim 6, wherein the portable electronic device is a mobile phone.

10. The sensor system according to claim 6, wherein the portable electronic device further comprises a storage unit electrically connected with the processing unit and adapted to store the digital signal.

11. The sensor system according to claim 6, wherein the target object is human body fluid.

12. The sensor system according to claim 6, wherein the physical and/or chemical data corresponds to blood glucose concentration, blood glucose level, lipid content, ion content in body fluid, blood oxygen content or urea nitrogen content.
